(19) United States
(12) Patent Application Publication
(10) Pub. No.: US 2003/0092171 A1
(43) Pub. Date: May 15, 2003

(54) Surface treatments for DNA processing devices

(76) Inventor: Steven Henck, Woodbridge, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/278,627

(22) Filed: Oct. 23, 2002

Related U.S. Application Data
(63) Continuation-in-part of application No. 09/192,605, filed on Nov. 16, 1998, now Pat. No. 6,475,722.

(51) Int. Cl. C12M 1/34; C23G 1/02

(52) U.S. Cl. 435/287.2; 134/3

(57) ABSTRACT

The present invention discloses methodologies for the treatment of the surface(s) of DNA processing devices so as to greatly reduce contamination with metal ions and semi-metal ions. These aforementioned surface treatments include an ammonium hydroxide and hydrogen peroxide wash, followed by a wash with EDTA which is followed by a wash with ammonium hydroxide and hydrogen peroxide. The present invention also discloses the fabrication of DNA processing devices utilizing surface(s) treated by the methods described. Such DNA processing devices include, for example, FORA, miniaturized electrophoresis and other DNA separation devices, such as miniaturized PCR reactors, and the like. Additionally, the methodologies and devices of the present invention are also applicable to the processing of nucleic acids, in general.

### RELATED APPLICATIONS

This application is a continuation-in-part of U.S. Ser. No. 09/192,605 and claims priority from U.S. Ser. No. 09/192,605 filed Nov. 16, 1998, now U.S. Pat. No. ______. The contents of U.S. Pat. No. ______ is incorporated herein by reference in its entirety.

### FIELD OF THE INVENTION

This invention is directed to novel processes for the treatment of devices of all types, and more particularly to processes for the treatment of miniaturized devices for the processing of biological materials or for the performance of biochemical reactions. Further, the invention is also directed to processes to minimize the effects of the exposed device surfaces on the process.

### BACKGROUND

A recent development in the field of biotechnology has been the development of miniaturized devices and systems for processing and analysis of DNA, proteins and other biological materials. Justifications for such devices include reduced cost, increased speed and reliability, distributed access (point-of-care diagnostics), decreased sample and reagent consumption and reduced waste generation.

An example of a miniaturized, massively parallel analysis device is set forth in commonly assigned U.S. application Ser. No. 10/104,280 and PCT publication WO 02/08700 filed Mar. 21, 2002, which is hereby incorporated by reference in its entirety. This application describes devices for the parallel analysis of DNA and proteins. In one embodiment, a device of this application includes a series of reactor chambers that are formed by etching the end of a fiber optic array bundle. These miniaturized features can be constructed by micromachining techniques, including the lithographic and etching methods developed in the semiconductor industry.

In commonly assigned U.S. application Ser. No. 09/192,605 filed Nov. 16, 1998, which is hereby incorporated by reference in its entirety, the need for surface treatments for surfaces created in micromachined DNA processing devices that prevent biochemical reactions and that inhibit DNA surface adsorption is discussed. Such an inhibition of adsorption is termed herein surface “passivation.” Due to their decreased dimensions the ratio of surface to volume in miniaturized or microfabricated DNA processing devices is greatly increased over their larger counter parts (M. A. Shoffner et al., 1996, Nucleic Research 24: 375). This increased surface-to-volume ratio increases the significance of effects of surface chemistry in such microfabricated devices. Metal ions in some substrates can compete for the natural metal cofactor, for example Mg⁺⁺ required for DNA polymerase activity, and interfere with their enzymatic activity. Although this problem is present in larger scale DNA processing devices, it is considerably exacerbated in micromachined devices with larger surface to volume ratios, and is a common problem in DNA processing systems such as PCR reactors, capillary and plate gel electrophoresis systems. It is well known in the art that DNA interacts strongly with and adheres to a number of surfaces (S. Hjerten 1985, J. Chromatogr., 347: 191). The hydrophilic phosphate groups and hydrophobic protonated bases mean that almost any surface is likely to interact.

DNA surface adherence was addressed for a microfabricated polymerase chain reaction (“PCR”) device (M. A. Shoffner et al., 1996, Nucleic Acids Research 24: 375). Several surface treatments were investigated in an attempt to find “PCR friendly” surfaces, including surface treatment by silanization followed by a polymer treatment, stoichiometric silicon nitride coating, and oxidization of the silicon surface. Only oxidization did not inhibit the PCR reaction. The inhibition of PCR reaction by the other treatments was presumed to have been the result of surface binding sites that non-specifically adsorbed molecules involved in the PCR reaction (J. Cheng, 1996, Nucleic Acids Research 24: 380).

The fiber optics which comprise the fiber optic array bundle device set forth in U.S. application Ser. No. 10/104,280 contain concentric layers of glasses that have been doped with various metal salts and oxides. The dopants change the index of refraction of the glasses such that the core of the fiber typically has a lower index of refraction than the cladding that surrounds it. Light is transmitted throughout the length of the fiber by internal reflections from the interface between the core and the cladding. The dopant metals, however, have been found to interfere with biochemical reactions occurring near the surface. This invention fulfills a need in the art for a treatment for the surfaces of biochemical apparatus, such as the fiber optic reactor array (FORA), to remove unnecessary contaminants particularly metal ions and semi-metal ions.

### SUMMARY OF THE INVENTION

In one aspect, the invention includes a method of treating a surface to enhance biological activities on or proximal to the surface comprising: (a) contacting the surface with a mixture solution comprising ammonium hydroxide and hydrogen peroxide; (b) contacting the surface with a solution comprising EDTA; and (c) contacting the surface with a solution comprising ammonium hydroxide and hydrogen peroxide to produce a treated surface. In one embodiment, the method further comprises one or more washing steps following each of the contacting steps wherein the washing step comprises contacting the surface with water for a period of at least five seconds.

In another aspect, the invention includes a method of treating a surface to enhance biological activities on or proximal to the surface comprising: (a) contacting the surface with a solution comprising EDTA; and (b) contacting the surface with a solution comprising ammonium hydroxide and hydrogen peroxide to produce a treated surface. In a preferred embodiment, the method further comprises one or more washing steps following each of the contacting steps wherein the washing step comprises contacting the surface with water for a period of at least five seconds. In another embodiment both of the methods described above, the treated glass has a surface composition with one or more of the following properties: (a) barium of no more than 1.6% by XPS; (b) aluminum of no more than 0.1% by XPS; and (c) boron of no more than 0.1% by XPS. Alternatively, the one or more surface has a composition of (a) barium of no more than 3.2% by XPS; (b) aluminum of no more than 0.2% by XPS; and (c) boron of no more than 0.2% by XPS.

In a further aspect, the invention includes a FORA with at least one surface having the following elemental compositions: (a) barium of no more than 1.6% by XPS; (b) aluminum of no more than 0.1% by XPS; and (c) boron of no more than 0.1% by XPS. In one embodiment, the FORA is made from X14 glass. Alternatively, the one or more surface has a composition of (a) barium of no more than 3.2% by XPS; (b) aluminum of no more than 0.2% by XPS; and (c) boron of no more than 0.2% by XPS.

In another aspect, the FORA is made from optical fiber comprising cladding glass wherein one exposed surface of the reaction core has been treated by the above mentioned methods. In a preferred embodiment, the FORA has one exposed surface comprising: (a) barium of no more than 1.6% by XPS; (b) aluminum of no more than 0.1% by XPS; and (c) boron of no more than 0.1% by XPS. Alternatively, the one or more surface has a composition of (a) barium of no more than 3.2% by XPS; (b) aluminum of no more than 0.2% by XPS; and (c) boron of no more than 0.2% by XPS.

In a further aspect, the invention includes a FORA wherein glass in the reaction core has the property of (a) barium of no more than 1.6% by XPS; (b) aluminum of no more than 0.1% by XPS; and (c) boron of no more than 0.1% by XPS. Alternatively, the one or more surface has a composition of (a) barium of no more than 3.2% by XPS; (b) aluminum of no more than 0.2% by XPS; and (c) boron of no more than 0.2% by XPS.

In another aspect, the invention includes a method of cleaning a surface of a biochemical apparatus, comprising: (a) contacting the surface with a solution of ammonium hydroxide and hydrogen peroxide; (b) contacting the surface with a solution of EDTA; and (c) contacting the surface with the solution of ammonium hydroxide and hydrogen peroxide. In a preferred embodiment, the solution of ammonium hydroxide and the hydrogen peroxide has a concentration of at least 1% ammonium hydroxide and at least 1% hydrogen peroxide. In another embodiment, the EDTA solution has a concentration between 0.1M and 1M. In a further embodiment, the biochemical apparatus is a FORA. In another embodiment, a surface contaminant is removed from the surface. The contaminant could be a metal ion or semi-metal ion. In one embodiment, the metal ion is selected from the group consisting of boron, sodium, magnesium, aluminum, titanium, niobium, barium and lanthanum. In another embodiment, the semi-metal ion is silicon.

In a further aspect, the invention includes a method of cleaning a surface of a biochemical apparatus, comprising: (a) contacting the surface with a solution of EDTA; and (b) contacting the surface with the solution of ammonium hydroxide and hydrogen peroxide. In one embodiment, at least one surface contaminant is removed from the surface. In another embodiment, the contaminant is a metal ion or semi-metal ion.

In another aspect, the invention provides a solution for treating the surface of a biochemical apparatus comprising ammonium hydroxide, hydrogen peroxide and EDTA in order to enhance a biochemical reaction.

In a further aspect, the invention includes a method for reducing the amount of metal ions present on a surface of a device for performing biochemical reactions, comprising: (a) optionally washing one or more surfaces with a solution of at least 1% ammonium hydroxide and at least 1% hydrogen peroxide; (b) washing the one or more surfaces with a solution of EDTA; (c) washing the one or more surfaces with a solution of at least 1% ammonium hydroxide and at least 1% hydrogen peroxide; thereby reducing the amount of metal ions bound to one or more surfaces used in a device for performing biochemical reactions.

In a preferred embodiment, the biochemical reactions include, but are not limited to: (i) DNA analysis (e.g., sequencing, separation, hybridization, electrophoresis; (ii) DNA processing (e.g., DNA replication, polymerase chain reaction (“PCR”), Reverse Transcription-based PCR (RT-PCR), ligase chain reaction (“LCR”), in vitro transcription and translation, strand exchange with or without enzymes); (iii) DNA modifications (e.g., end- or internal-labeling, phosphorylation, de-phosphorylation, digestion, ligation, multiplex formation for strand identification); (iv) DNA packaging (e.g., linking to form higher ordered structures); and (v) DNA extraction. In a preferred embodiment, the EDTA solution has a concentration between 0.1M and 1M. In another embodiment, the method further comprises one or more washing steps following each of the contacting steps wherein the washing step comprises contacting the surface with water for a period of at least five seconds. In a further embodiment, one or more surface has a composition comprising: (a) barium of no more than 1.6% by XPS; (b) aluminum of no more than 0.1% by XPS; and (c) boron of no more than 0.1% by XPS. Alternatively the one or more surface has a composition of (a) barium of no more than 3.2% by XPS; (b) aluminum of no more than 0.2% by XPS; and (c) boron of no more than 0.2% by XPS. The invention also includes an apparatus that has been treated by any of the above described methods.

In another aspect, the invention includes a method for removing metal ion or semi-metal ion contaminants on one or more surfaces used in a device, the method comprising: (a) washing the one or more surfaces with an EDTA solution; and (b) washing the one or more surfaces with an alkaline solution comprising an oxidizing agent, wherein said solution is at a temperature between room temperature and 75° C. thereby reducing the amount of metal ion or semi-metal ion contaminants bound to one or more surfaces of the device.

In a further aspect, the invention includes a method for reducing the amount of metal ion or semi-metal ion contaminants bound to one or more surfaces used in a device, the method comprising: (a) washing the one or more surfaces with an alkaline solution comprising an oxidizing agent, wherein said solution is at a temperature between room temperature and 75° C.; (b) washing the one or more surfaces with an EDTA solution; and (c) repeating step (a) thereby reducing the amount of metal ion or semi-metal ion contaminants bound to one or more surfaces used in the device.

In another aspect, the invention includes a method for minimizing metal ion or semi-metal ion contaminants on a surface, the method comprising: (a) providing a device comprising a surface for performing a biochemical reaction; (b) treating the surface with an EDTA solution; (c) treating the surface with an alkaline solution comprising an oxidizing agent, wherein said solution is at a temperature between room temperature and 75° C. thereby removing metal ion or semi-metal ion contaminants bound to the surface.

In a further aspect, the invention includes a method for minimizing contamination with metal ions or semi-metal ions to a surface, the method comprising: (a) providing a device comprising a surface; (b) treating said surface with an alkaline solution comprising an oxidizing agent, wherein said solution is at a temperature between room temperature and 75° C.; (c) treating said surface with an EDTA solution; (d) repeating step (b) thereby minimizing contamination with metal ions or semi-metal ions on the surface of the device.

### BRIEF DESCRIPTION OF THE DRAWINGS

FIG. 1: illustrates an exemplary embodiment of a microfabricated device for DNA processing to which the instant invention is applicable (e.g., fiber optic reactor array (FORA).

FIG. 2: Panel A illustrates emission spectra observed from the surface of a X-14 FORA with various surface treatments;

### DETAILED DESCRIPTION OF INVENTION

This invention provides methods for the surface treatment of DNA processing devices as well as devices made by such surface treatments. These treatments are particularly useful for devices which may contact biochemical reaction mediums and are susceptible to contamination by metal salts and oxides and other contaminants. In such devices, it is particularly advantageous to reduce the amount of contaminating species.

Accordingly, the methods of the invention provide for the treatment of any surface which shows an ability to inhibit a biological reaction. The method is applied to the surface to create a “treated surface.” After using the methods of the invention, the treated surface displays a reduced inhibition of a biological reaction that occurs in the proximity of (on or near) the surface. In the method, a surface to be treated is contacted with a cleaning solution, containing ammonium hydroxide and hydrogen peroxide. Subsequently, the surface is contacted with an organic metal chelating reagents such as ethylenediaminetetraacetic acid (EDTA). The surface is then treated again with ammonium hydroxide and hydrogen peroxide.

These treatments are advantageously applied to nucleic acid processing devices of all sizes and many types. Although biochemical process inhibition is exacerbated in micromachined devices, it is also present in larger scale nucleic acid processing devices. Nucleic acid processing devices include such commonly used instruments as electrophoresis devices of all configurations, including slab, capillary, and micromachined. They further include processing devices such as reactors for performing PCR reactions, sequencing reactions, and other enzymatic reactions such as restriction endonuclease digestion, ligation, and so forth. The subsequent sections introduce the invention by describing, exemplary processing devices, exemplary surface treatment processes. The particular conditions suitable for surface treatment are described.

Exemplary Processing Devices

This subsection describes exemplary DNA separation devices and an exemplary DNA processing reactor configuration. One of ordinary skill within the art will readily appreciate how the methods of this invention can be applied to most types of DNA processing devices possessing surfaces which are amenable to the treatments disclosed herein.

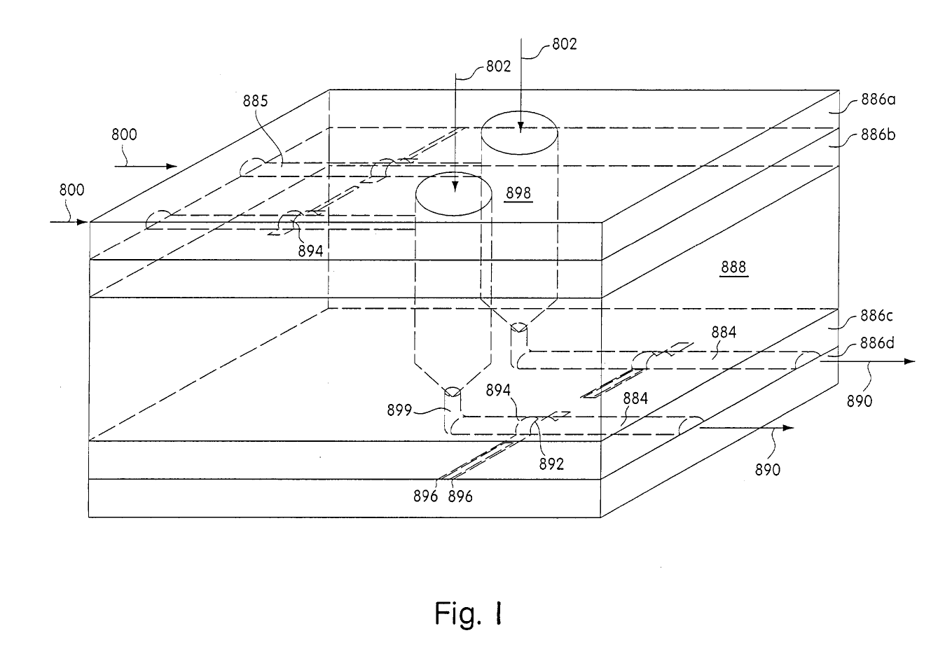

FIG. 1 illustrates a section of an exemplary nucleic acid processing reactor array. Illustrated are two micro-reactors (898), which are formed in substrate (888). These reactors hold reagents during the nucleic acid processing reactions (e.g., DNA sequencing reactions). The substrate (888) is typically comprised of glass, and the reactors (898) are typically microfabricated by either etching or drilling processes. The microreactors are supplied with reagents through both the top ports (802) and the inlet ports (800), which are conducted via inlet capillary channels (894 and 895) to the microreactors. Products are conducted to the outlet ports (890) by the outlet capillary channels (894) which are controlled by assemblies (894, 895 and 896), which may, preferably, be electrothermal microvalves. Inlet and outlet capillary channels are formed in substrates 866a and 866b and in substrates 866c and 866d, respectively. These substrates are typically silicon, and the capillaries are formed by micro-lithographic processes. The capillaries are then advantageously coated with a layer of, e.g., thermally grown silicon oxide to render them more inert. Design and fabrication of such a micro-reactor array are further described in, e.g., PCT publication WO 96/35810.

Also in such an apparatus, medium containing not only DNA, but also, for example, enzymes, reagents, labeled oligomers, and so forth, comes in contact with many device components. In particular, the capillaries have a higher surface to volume ratio and may be coated with thermally grown oxide. They are likely to undesirably capture molecules if they are reactive. It is also even more advantageous to reduce adherence, and especially DNA adherence, in such an apparatus.

This invention could be used to treat an apparatus for sequencing nucleic acids in order to enhance biochemical reactions. This apparatus generally comprises one or more reaction chambers for conducting a sequencing reaction, means for delivering reactants to and from the reaction chamber(s), and means for detecting a sequencing reaction event. The apparatus could also include a reagent delivery cuvette containing a plurality of cavities on a planar surface. The apparatus is connected to at least one computer for controlling the individual components of the apparatus and for storing and/or analyzing the information obtained from detection of the sequence reaction event.

This sequencing apparatus also contains one or more reaction chambers that are arranged in the form of an array on an inert substrate material, also referred to herein as a “solid support”, that allows for combination of the reactants in a sequencing reaction in a defined space and for detection of the sequencing reaction event. Thus, as used herein, the terms “reaction chamber” or “analyte reaction chamber” refer to a localized area on the substrate material that facilitates interaction of reactants, e.g., in a nucleic acid sequencing reaction. The sequencing reactions contemplated preferably occur on numerous individual nucleic acid samples in tandem, in particular simultaneously sequencing numerous nucleic acid samples derived from genomic and chromosomal DNA. The sequencing apparatus therefore preferably comprises an array having a sufficient number of reaction chambers to carry out such numerous individual sequencing reactions. In one embodiment, the array comprises at least 1,000 reaction chambers. In another embodiment, the array comprises greater than 400,000 reaction chambers, preferably between 400,000 and 20,000,000 reaction chambers. In a more preferred embodiment, the array comprises between 1,000,000 and 16,000,000 reaction chambers.

Solid Support Material for Sequencing Apparatus

Any material can be used as the solid support material, as long as the surface allows for stable attachment of the primers and detection of nucleic acid sequences. The solid surface may be of any shape such as planar, spherical (bead), tubular, rod like, particles or irregularly shaped. In a preferred embodiment, the solid support material can be planar or can be cavitated. For example, the solid support may be a cavitated terminus of a fiber optic. The cavitated surface may be a microwell etched, molded, stamped or otherwise micromachined into the surface. Techniques for the fabrication of such solid supports are commonly used in the construction of microelectromechanical systems. See e.g., Rai-Choudhury, HANDBOOK OF MICROLITHOGRAPHY, MICROMACHINING, AND MICROFABRICATION, VOLUME I: MICROLITHOGRAPHY, Volume PM39, SPIE Press (1997); Madou, CRC Press (1997), Aoki, Biotech. Histochem. 67: 98-9 (1992); Kane et al., Biomaterials. 20: 2363-76 (1999); Deng et al., Anal. Chem. 72:3176-80 (2000); Zhu et al., Nat. Genet. 26:283-9 (2000). In a preferred embodiment, the solid support may be made of a material that is mostly optically transparent material such as glass. It should be the noted that the optically transparent material should have sufficient transparency to allow optical monitoring of any reaction. Absolute transparency is not required. For example, a transmission of 10% is sufficient if a biological reaction may be monitored by detecting only 10% of the photon emission.

An array of attachment sites on an optically transparent solid support can be constructed using lithographic techniques commonly used in the construction of electronic integrated circuits as described in, for example, U.S. Pat. Nos. 5,143,854, 5,445,934, 5,744,305, and 5,800,992; Chee et al., Science 274: 610-614 (1996); Fodor et al., Nature 364: 555-556 (1993); Fodor et al., Science 251: 767-773 (1991); Gushin, et al., Anal. Biochem. 250: 203-211 (1997); Kinosita et al., Cell 93: 21-24 (1998); Kato-Yamada et al., J. Biol. Chem. 273: 19375-19377 (1998); and Yasuda et al., Cell 93: 1117-1124 (1998). Photolithography and electron beam lithography sensitize the solid support or substrate with a linking group that allows attachment of a modified biomolecule (e.g., proteins or nucleic acids). See e.g., Service, Science 283: 27-28 (1999); Rai-Choudhury, HANDBOOK OF MICROLITHOGRAPHY, MICROMACHINING, AND MICROFABRICATION, VOLUME I: MICROLITHOGRAPHY, Volume PM39, SPIE Press (1997). Alternatively, an array of sensitized sites can be generated using thin-film technology as described in Zasadzinski et al., Science 263: 1726-1733 (1994).

Fiber Optic Substrate Arrays

The substrate material is preferably made of a material that facilitates detection of the reaction event. For example, in a typical sequencing reaction, binding of a dNTP to a sample nucleic acid to be sequenced can be monitored by detection of photons generated by enzyme action on phosphate liberated in the sequencing reaction. Thus, having the substrate material made of a transparent or light conductive material facilitates detection of the photons.

In some embodiments, the solid support can be coupled to a bundle of optical fibers that are used to detect and transmit the light product. The total number of optical fibers within the bundle may be varied so as to match the number of individual reaction chambers in the array utilized in the sequencing reaction. The number of optical fibers incorporated into the bundle is designed to match the resolution of a detection device so as to allow 1:1 imaging. The overall sizes of the bundles are chosen so as to optimize the usable area of the detection device while maintaining desirable reagent (flow) characteristics in the reaction chamber. Thus, for a 4096×4096 pixel CCD (charge-coupled device) array with 15 μm pixels, the fiber bundle is chosen to be approximately 60 mm×60 mm or to have a diameter of approximately 90 mm. The desired number of optical fibers are initially fused into a bundle or optical fiber array, the terminus of which can then be cut and polished so as to form a “wafer” of the required thickness (e.g., 1.5 mm). The resulting optical fiber wafers possess similar handling properties to that of a plane of glass. The individual fibers can be any size diameter (e.g., 3 μm to 100 μm).

In some embodiments two fiber optic bundles are used: a first bundle is attached directly to the detection device (also referred to herein as the fiber bundle or connector) and a second bundle is used as the reaction chamber substrate (the wafer or substrate). In this case the two are placed in direct contact, optionally with the use of optical coupling fluid, in order to image the reaction centers onto the detection device. If a CCD is used as the detection device, the wafer could be slightly larger in order to maximize the use of the CCD area, or slightly smaller in order to match the format of a typical microscope slide—25 mm×75 mm. The diameters of the individual fibers within the bundles are chosen so as to maximize the probability that a single reaction will be imaged onto a single pixel in the detection device, within the constraints of the state of the art. Exemplary diameters are 6-8 μm for the fiber bundle and 6-50 μm for the wafer, though any diameter in the range 3-100 μm can be used. Fiber bundles can be obtained commercially from CCD camera manufacturers. For example, the wafer can be obtained from Incom, Inc. (Charlton, Mass.) and cut and polished from a large fusion of fiber optics, typically being 2 mm thick, though possibly being 0.5 to 5 mm thick. The wafer has handling properties similar to a pane of glass or a glass microscope slide.

Reaction chambers can be formed in the substrate made from fiber optic material. The surface of the optical fiber is cavitated by treating the termini of a bundle of fibers, e.g., with acid, to form an indentation in the fiber optic material. Thus, in one embodiment cavities are formed from a fiber optic bundle, preferably cavities can be formed by etching one end of the fiber optic bundle. Each cavitated surface can form a reaction chamber. Such arrays are referred to herein as fiber optic reactor arrays or FORA. The indentation ranges in depth from approximately one-half the diameter of an individual optical fiber up to two to three times the diameter of the fiber. Cavities can be introduced into the termini of the fibers by placing one side of the optical fiber wafer into an acid bath for a variable amount of time. The amount of time can vary depending upon the overall depth of the reaction cavity desired (see e.g., Walt, et al., 1996. Anal. Chem. 70: 1888). A wide channel cavity can have uniform flow velocity dimensions of approximately 14 mm×43 mm. Several methods are known in the art for attaching molecules (and detecting the attached molecules) in the cavities etched in the ends of fiber optic bundles. See, e.g., Michael, et al., Anal. Chem. 70: 1242-1248 (1998); Ferguson, et al., Nature Biotechnology 14: 1681-1684 (1996); Healey and Walt, Anal. Chem. 69: 2213-2216 (1997). A pattern of reactive sites can also be created in the microwell, using photolithographic techniques similar to those used in the generation of a pattern of reaction pads on a planar support. See, Healey, et al., Science 269: 1078-1080 (1995); Munkholm and Walt, Anal. Chem. 58: 1427-1430 (1986), and Bronk, et al., Anal. Chem. 67: 2750-2757 (1995).

The opposing side of the optical fiber wafer (e.g., the non-etched side) is typically highly polished so as to allow optical-coupling (e.g., by immersion oil or other optical coupling fluids) to a second, optical fiber bundle. This second optical fiber bundle exactly matches the diameter of the optical wafer containing the reaction chambers, and serve to act as a conduit for the transmission of light product to the attached detection device, such as a CCD imaging system or camera.

In one embodiment, the fiber optic wafer is thoroughly cleaned by a series of washes. For example, the first step may be a wash using an aqueous solution containing 5% H₂O₂ and 5% NH₄OH (volume:volume). This second step may be six rinses in deionized water. The third step may be a wash in 0.5M EDTA. The fourth step may be another six deionized water rinses. The fifth step may be another wash with an aqueous solution containing 5% H₂O₂ and 5% NH₄OH (volume:volume). The sixth step may be six rinses in deionized water. The individual washes may be, for example, one-half hour incubations in each wash solution. While each step is disclosed separately for clarity, all the steps may be performed continuously. For example, the surface to be treated may be under continuous flow with solutions changed automatically. Alternatively, the surface may be dipped or sprayed with solution under automation. In a preferred embodiment, the fiber optic wafer is treated with a high pH cleaning solution of the invention including washes with ammonium hydroxide and hydrogen peroxide followed by a treatment with EDTA and followed by a second ammonium hydroxide and hydrogen peroxide wash.

The methods of the invention are suited for surfaces of devices that are exposed to the solutions used to produce reactions to analyze DNA. The method is particularly useful for devices with surfaces that may undesirably release reactive contaminants. Accordingly, surface treatments that generally obviate or eliminate interference with the reactions has been a long felt need which is achieved by the instant invention.

General Surface Treatments

Surface treatments which are useful for treating microfabricated and larger nucleic acid processing devices generally include: (i) the deposition of certain films or coatings of controlled and desirable properties or (ii) the utilization of various surface washes designed to remove unwanted materials and leave the indigenous surface groups in a controlled state. The deposited films are applicable to a wider variety of surfaces including, but not limited to, silicon, silicon oxides, glasses, metals, plastics and other similar materials; where the surface washings are applicable, preferably, to surfaces comprised of silicon or silicon oxide. The following subsection will more-fully discuss both of these aforementioned types of surface treatments.

Suitable materials for coating the FORA include, for example, plastics such as polystyrene. Plastics are readily adaptable for coating purposes and, for example, may be preferably spin-coated or sputtered in a thickness of about 0.1 μm. Other materials for coating the array include gold which can also be coated in a thickness of 0.1 μm. The gold may be further treated with adsorbed self-assembling monolayers of long chain thiol alkanes. Biotin is then coupled covalently to the long chain thiol bound surface and saturated with a biotin-binding protein (e.g. streptavidin or avidin). The robustness of the chemically bonded self-assembling monolayers combined with the high affinity of the streptavidin-biotin linker layer provide a flexible platform for assay development.

Additional coating materials can also include those systems used to attach an anchor primer to a substrate. Organosilane reagents, which allow for direct covalent coupling of proteins via amino, sulfhydryl or carboxyl groups, can also be used to coat the array. Additional coating substances include photoreactive linkers, e.g. photobiotin, (Amos et al., “Biomaterial Surface Modification Using Photochemical Coupling Technology,” in Encyclopedic Handbook of Biomaterials and Bioengineering, Part A: Materials, Wise et al. (eds.), New York, Marcel Dekker, pp. 895926, 1995) which may be synthesized or are available commercially.

Additional coating materials include hydrophilic polymer gels (polyacrylamide, polysaccharides), which preferably may be applied by polymerizing the material directly on the surface. Alternatively, the polymer chains may be covalently attached to the surface after polymerization (Hjerten, J. Chromatogr. 347,191 (1985); Novotny, Anal. Chem. 62,2478 (1990). Other suitable polymers include pluronic polymers (triblock copolymers, e.g. PPO-PEO-PPO, also known as F-108) which can be specifically adsorbed to either polystyrene or silanized glass surfaces (Ho et al., Langmuir 14:3889-94, 1998) or passively adsorbed onto layers of biotin-binding proteins. To facilitate the coating with these polymers, the surface can also be coated with an epoxide which allows the coupling of reagents via an amine linkage.

In addition, any of the above materials can be derivatized with one or more functional groups, commonly known in the art for the immobilization of enzymes and nucleotides, e.g. metal chelating groups (e.g. nitrilo triacetic acid, iminodiacetic acid, pentadentate chelator), which will bind polyhistidine-tagged (4, 5, 6, 7, 8, 9, or 10 histidine) proteins and nucleic acids.

In another embodiment, surface coatings can be used to increase the number of available binding sites for subsequent treatments (e.g., attachment of enzymes discussed below) beyond the theoretical binding capacity of a two dimensional surface.

In a preferred embodiment, the individual optical fibers utilized to generate the fused optical fiber bundle/wafer are larger in diameter (e.g., 6 μm to 12 μm) than those utilized in the optical imaging system (e.g., 3 μm). Thus, several of the optical imaging fibers can be utilized to image a single reaction site.

Surface Washings

It has been demonstrated that certain basic surface washings advantageously reduce contamination of surfaces including a reduction in DNA adherence. Such basic surface washings are believed to function by leaving reactive sites on a surface terminated by neutral, hydrophilic hydroxyl groups. DNA has reduced affinity to such neutral, hydrophilic, basic surfaces. Suitable basic wash solutions contain an alkalinizing agent in a solvation fluid which cause the pH or the solution to be 8, 10, 12, 14, or higher. Suitable alkalinizing agents include ammonium hydroxide (NH₄OH), potassium hydroxide (KOH), sodium hydroxide (NaOH), or other bases. Suitable solvation fluids include water, and alcohols (e.g., methanol (MeOH) or ethanol (EtOH)) and, in particular an EtOH/KOH solution, are adaptable to the present invention. An optional, but preferred embodiment includes oxidizing agents in order to oxidize (and thereby remove) residual organic contaminants left from any previous microfabrication steps. Suitable oxidizing agents include peroxides, chlorates, perchlorates, nitrates, permanganates, and so forth. Most preferred solvation, alkalinizing, and oxidizing agents are easily volatile without leaving any residues.

Preferred surface washing solutions include aqueous (solvation agent) solutions of ammonium hydroxide or sodium hydroxide (alkalinizing agents) in combination with hydrogen peroxide (H₂O₂) (oxidizing agent). A most preferred surface washing solution includes approximately 4 parts water, approximately 1 part 30% hydrogen peroxide, and approximately 1 part 30% ammonium hydroxide. This latter solution is most preferred because, first, all its components are volatile and leave no residue on a surface, and because, second, it is strongly oxidizing and is capable of oxidizing and removing organic surface contaminants. While the solution is most preferred in part because it acts rapidly, a more dilute solution (e.g., 1:2, 1:4, 1:5, 1:10, 1:20, 1:50 or 1:100 dilution with water) may be used if speed is not important. A broad range of compositions are most preferred, as long as sufficient ammonium hydroxide is present so that the wash solution is sufficiently basic and sufficient hydrogen peroxide is present so that expected organic surface contaminants can be oxidized. The reagents are preferably of such a purity (e.g., reagent grade, analytical grade or better) such that they leave no contaminants themselves upon volatilizing from a surface.

Device surfaces to be treated are exposed to the wash solution at room temperature for a time period preferably from 1 minute to 1 hour with a 10 minute wash being the most preferred embodiment. These times are optimal, in part, because of efficiency considerations. However, if a more dilute wash solution, as listed above, is used, the incubation time may be increased to 2 hours, 4 hours, 8 hours or overnight. The optimal time and concentration may be determined with the parameters set forth in this disclosure. For example, if the solution is diluted two fold or four fold, the incubation time may be increased two fold or four fold. The present invention is also adaptable to shorter or longer exposures, as well as to exposures at elevated temperatures, up to the actual boiling point of the wash solution. Following this treatment, the surfaces are then rinsed with water, preferably deionized water, so as to remove any remaining wash solution. The equipment utilized for exposing surfaces is typically either an immersion bath (with or without ultrasonic agitation) or a spin-spray device.

Semiconductor processing also uses surface washings and chemical cleanings, although typically with markedly different processing parameters than disclosed herein in the preferred embodiment. See e.g., Runyan, et al., 1990. Semiconductor Integrated Circuit Processing Technology pp. 99-104 (Addison-Wesley; Reading, Mass.). Surface exposure to wash solutions is typically at a temperature ranging from 75° C. to near 100° C. Sodium hydroxide solutions are not typically utilized in semiconductor processing, due to the potential contaminating effect of this alkali metal (e.g., sodium) including reduction of integrated circuit oxide field and charge build up in the oxide insulator. Surface washings, or chemical cleaning, are a common type of surface treatment. Semiconductor processing routinely makes use of chemical cleaning to maintain the purity and quality of the material. Aqueous mixtures of ammonium hydroxide and hydrogen peroxide are typically used as part of a cleaning routine referred to as the “RCA Clean” (see e.g., Kern & Puotinen, 1970. RCA Review 31:187, which is hereby incorporated by reference in its entirety). However, in semiconductor processing, a final acidic wash is generally always performed in order to remove contaminating metallic species. Such a final acidic wash would destroy the hydroxylated surface which the present invention is dependent upon, thus resulting in the formation of a charged surface capable of interacting with DNA (W. Kern and D. A. Puotinen, 1970, RCA Review, 31:187). An RCA clean is generally used to remove residual organic species and certain metals from surfaces. The equipment used for cleaning is typically either immersion baths, with or without ultrasonic agitation) or a spin-spray device, or spin-spray.

Ethylenediaminetetraacetic acid, EDTA, is commonly used as a metal chelating agent. It is known to form water-soluble salts containing from one to four alkali metal cations.

Evaluation of Preferred Surface Treatments of this Invention

In accordance with the present invention the approach is to treat the surfaces which exhibit unwanted interactions with a high pH cleaning solution such as 1:1 ammonium hydroxide (28% by volume) and hydrogen peroxide (30% by volume) Similarly, aqueous solutions of ammonium hydroxide or sodium hydroxide should also be effective. Additionally, the surfaces are treated with a 0.5 molar aqueous EDTA solution. The ammonium hydroxide/hydrogen peroxide treatment is similar to a process used in semiconductor processing known as RCA standard clean solution 1, SC1, although that treatment is typically performed with more dilute solutions and at elevated temperatures, ˜75° C.

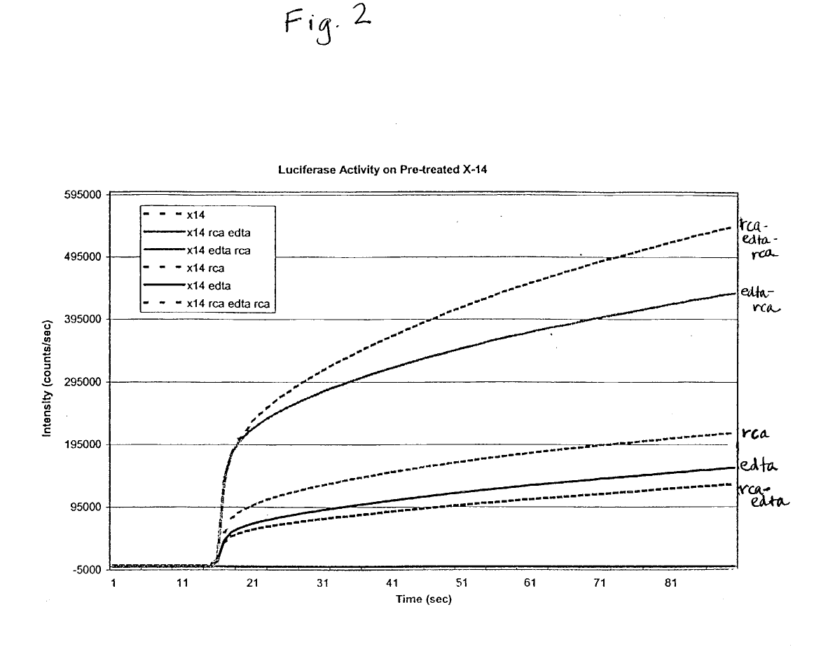

The device surface to be treated is exposed to the solutions at room temperature for a time period preferably from 30 seconds to 1 hour or longer with 10 minutes most preferred, although shorter or longer exposures, are possible as are exposures at elevated temperatures, or with solutions of higher or lower concentration. Following each treatment the surfaces are rinsed with water to remove the solution. FIG. 2 illustrates the beneficial effect of treating FORA surfaces with such an approach.

The present invention is further described in the following specific examples, which are in no way intended to limit the scope of the invention disclosed herein.

### EXAMPLES

### Example 1

### Analysis of the FORA Core and Surface

X-ray photoelectron spectroscopy (XPS) was utilized to view the core of the FORA. XPS measures characteristic energies and intensities of photoelectrons ejected from the surface of a solid material irradiated by soft x-rays. A 10 μm spot size at a 45 degree takeoff angle was used to examine the FORA core. The control sample was an untreated glass slide. For the etched sample, shallow etched wells (ca. 15 μm) at 50 μm pitch were used to allow for an unobstructed view of the core. The treatment of this instant invention was performed for the RER sample (e.g., RCA cleaned/EDTA treated/RCA cleaned). The Calculated sample refers to the theoretical composition of glass as stated by the vendor.

TABLE 1
XPS analysis of the D-14 FORA core.

| | B | C | O | Na | Mg | Al | Si | Ti | Nb | Ba | La |
|---|---|---|---|---|---|---|---|---|---|---|---|
| Control | 7.7 | 19.5 | 53.0 | — | — | 2.0 | 4.6 | 4.9 | 2.2 | 2.9 | 3.3 |
| Etched | 1.2 | 20.7 | 56.1 | — | — | — | 5.3 | 8.0 | 6.0 | 0.7 | 2.0 |
| RER | 0.1 | 16.8 | 57.2 | — | — | 0.1 | 10.4 | 7.7 | 3.0 | 1.6 | 3.2 |
| Calculated | 10.1 | 0 | 62 | — | — | — | 5.9 | 7.5 | 2.1 | 7.0 | 5.4 |

— indicates that the level is below detection limits.

As shown in Table 1, the major difference between the RER, sample and the Control is the amount of boron (B), barium (Ba) and aluminum (Al). This data shows that the RER treatment has removed metallic elements and changed the surface composition of the FORA. These results are very important in that B, Ba and Al are all significant inhibitors of biological reactions. Therefore, the RER cleaning will be useful in removing surface contaminants which would adversely effect a variety of biochemical reactions.

FIG. 2 illustrates the beneficial effect of treating a fiber optic reactor array surface with the RER cleaning method. Initially, five 0.5 cm squares of X-14 glass were placed in separate 15 ml Falcon tubes. For the RCA treatments, the pieces were soaked in 5 ml of RCA solution (1:1 NH₄OH and H₂O₂) at room temperature for 10 minutes. Following the treatment, the pieces were rinsed three times with deionized water. For the EDTA treatments, the pieces were soaked in 0.5 M EDTA for 10 minutes. Following the treatment, the pieces were rinsed three times with deionized water. Combinations and permutations of the order of the treatments was modulated as shown. For example, one preferred treatment includes the RCA treatment followed by the EDTA treatment followed by the RCA treatment. Another preferred example is the EDTA treatment followed by the RCA treatment. The metric to judge the efficacy of the treatments was the measurement of the luminescent emission of luciferase bound to the surface as described herein.

The surface of the X-14 FORA was assayed by determining the quantity and activity of luciferase bound to the surface of the glass. After the substrates were treated and washed, the surfaces were assayed. The glass was coated with a functionalized linker by soaking for one hour in 0.33 mg/μl Strept Avidin. They were then washed three times in a buffer consisting of 100 mM NaHCO₃. They were then placed in 1 ml of 0.05 mg/ml biotinylated Luciferase. They were then washed 6 times in Assay Buffer. Following the rinse the sample was placed in a tube containing 100 μl of 1 μM ATP; 5 μl of Pyrosequencing Substrate; and 300 μl of Assay Buffer. The luminescent emission was then measured.

The results of this experiment are shown in FIG. 2. In FIG. 2A, X14 is an untreated control chip. “X14 edta” received only the EDTA treatment and shows slightly higher luminescent intensity. “X14 rca” received only the RCA treatment and shows nearly double the amount of luminescent intensity than the control. “X14 rca edta” received the RCA treatment followed by the EDTA treatment; its luminescent intensity is negligible. “X14 edta rca” received the EDTA treatment followed by the RCA treatment; its luminescent intensity is nearly four times higher than the control. Finally, “x14 rca edta rca” received the RCA treatment followed by the EDTA treatment followed by the RCA treatment. Its luminescent intensity is nearly five times greater than the control.

The results of this experiment were surprising in that they clearly show that surface cleaning with RER (RCA, EDTA, RCA) or EDTA followed by RCA prior to addition of enzymes greatly enhances the activity of the enzymes on the surface of the glass as compared to no treatment or treatment with RCA, EDTA, or RCA followed by EDTA. The dramatic increase in luciferase activity on the glass treated with RER helps to demonstrate the usefulness in this treatment for removing contaminants from a wide range of surfaces.

Although the invention has been described with reference to be limited in scope by the specific embodiments described herein, this description is not meant to be construed in a limiting sense. Various modifications of the invention in addition to those described embodiments, as well as alternative embodiments, will be apparent to persons skilled in the art from. It is, therefore, contemplated that the foregoing description and accompanying figures. Such appended claims will cover all modifications are intended to that fall within the scope of the appended invention.

All patents, patent applications and references cited in this disclosure are incorporated herein by reference in their entireties.

We claim:

1. A method of treating a surface to enhance biological activities on or proximal to the surface comprising:
(a) contacting the surface with a mixture solution comprising ammonium hydroxide and hydrogen peroxide;
(b) contacting the surface with a solution comprising EDTA; and
(c) contacting the surface with a solution comprising ammonium hydroxide and hydrogen peroxide to produce a treated surface.

2. The method of claim 1 further comprising one or more washing steps following each of the contacting steps wherein the washing step comprises contacting the surface with water for a period of at least five seconds.

3. A method of treating a surface to enhance biological activities on or proximal to the surface comprising:
(a) contacting the surface with a solution comprising EDTA; and
(b) contacting the surface with a solution comprising ammonium hydroxide and hydrogen peroxide to produce a treated surface.

4. The method of claim 3 further comprising one or more washing steps following each of the contacting steps wherein the washing step comprises contacting the surface with water for a period of at least five seconds.

5. The method of claim 1 or 3 wherein the treated glass has a surface composition with one or more of the following properties:
(a) barium of no more than 1.6% by XPS;
(b) aluminum of no more than 0.1% by XPS; and
(c) boron of no more than 0.1% by XPS.

6. A FORA with at least one surface having the following elemental compositions:
(a) barium of no more than 1.6% by XPS;
(b) aluminum of no more than 0.1% by XPS; and
(c) boron of no more than 0.1% by XPS.

7. The FORA of claim 6 wherein the FORA is made from X14 glass.

8. A FORA made from optical fiber comprising cladding glass and wherein one exposed surface of the reaction core has been treated by the method of claim 1 or 3.

9. The FORA of claim 8 wherein said one exposed surface comprises:
(a) barium of no more than 1.6% by XPS;
(b) aluminum of no more than 0.1% by XPS; and
(c) boron of no more than 0.1% by XPS.

10. A FORA wherein glass in the reaction core has the property of
(a) barium of no more than 1.6% by XPS;
(b) aluminum of no more than 0.1% by XPS; and
(c) boron of no more than 0.1% by XPS.

11. A method of cleaning a surface of a biochemical apparatus, comprising:
(a) contacting the surface with a solution of ammonium hydroxide and hydrogen peroxide;
(b) contacting the surface with a solution of EDTA; and
(c) contacting the surface with the solution of ammonium hydroxide and hydrogen peroxide.

12. The method of claim 11 wherein the solution of ammonium hydroxide and the hydrogen peroxide have a concentration of at least 1% ammonium hydroxide and at least 1% hydrogen peroxide.

13. The method of claim 11 wherein the EDTA solution has a concentration between 0.1M and 1M.

14. The method of claim 11 wherein the biochemical apparatus is a FORA.

15. The method of claim 11 wherein a surface contaminant is removed from the surface.

16. The method of claim 15 wherein the contaminant is a metal ion or semi-metal ion.

17. The method of claim 16 wherein the metal ion is selected from the group consisting of boron, sodium, magnesium, aluminum, titanium, niobium, barium and lanthanum.

18. The method of claim 16 wherein the semi-metal ion is silicon.

19. A method of cleaning a surface of a biochemical apparatus, comprising:
(a) contacting the surface with a solution of EDTA; and
(b) contacting the surface with the solution of ammonium hydroxide and hydrogen peroxide.

20. The method of claim 19 wherein the solution of ammonium hydroxide and the hydrogen peroxide have a concentration of at least 1% ammonium hydroxide and at least 1% hydrogen peroxide.

21. The method of claim 19 wherein the EDTA solution has a concentration between 0.1M and 1M.

22. The method of claim 19 wherein the biochemical apparatus is a FORA.

23. The method of claim 19 wherein at least one surface contaminant is removed from the surface.

24. The method of claim 23 wherein the contaminant is a metal ion or semi-metal ion.

25. The method of claim 24 wherein the metal ion is selected from the group consisting of boron, sodium, magnesium, aluminum, titanium, niobium, barium and lanthanum.

26. The method of claim 24 wherein the semi-metal ion is silicon.

27. A solution for treating the surface of a biochemical apparatus in order to enhance a biochemical reaction, comprising ammonium hydroxide, hydrogen peroxide and EDTA.

28. A method for reducing the amount of metal ions present on a surface of a device for performing biochemical reactions, comprising:
(a) optionally washing one or more surfaces with a solution of at least 1% ammonium hydroxide and at least 1% hydrogen peroxide;
(b) washing the one or more surfaces with a solution of EDTA;
(c) washing the one or more surfaces with a solution of at least 1% ammonium hydroxide and at least 1% hydrogen peroxide; thereby reducing the amount of metal ions bound to one or more surfaces used in a device for performing biochemical reactions.

29. The method of claim 28 wherein the biochemical reaction is selected from the group consisting of DNA analysis, DNA processing, DNA modifications and DNA packaging.

30. The method of claim 28 wherein the EDTA solution has a concentration between 0.1M and 1M.

31. The method of claim 28 further comprising one or more washing steps following each of the contacting steps wherein the washing step comprises contacting the surface with water for a period of at least five seconds.

32. The method of claim 28 wherein the one or more surface has a composition comprising:
(a) barium of no more than 1.6% by XPS;
(b) aluminum of no more than 0.1% by XPS; and
(c) boron of no more than 0.1% by XPS.

33. An apparatus treated by the method as in claims 3, 19 or 28.

34. A method for removing metal ion or semi-metal ion contaminants on one or more surfaces used in a device, the method comprising:
(a) washing the one or more surfaces with an EDTA solution; and
(b) washing the one or more surfaces with an alkaline solution comprising an oxidizing agent, wherein said solution is at a temperature between room temperature and 75° C. thereby reducing the amount of metal ion or semi-metal ion contaminants bound to one or more surfaces of the device.

35. A method for reducing the amount of metal ion or semi-metal ion contaminants bound to one or more surfaces used in a device, the method comprising:
(a) washing the one or more surfaces with an alkaline solution comprising an oxidizing agent, wherein said solution is at a temperature between room temperature and 75° C.;
(b) washing the one or more surfaces with an EDTA solution; and
(c) repeating step (a) thereby reducing the amount of metal ion or semi-metal ion contaminants bound to one or more surfaces used in the device.

36. A method for minimizing metal ion or semi-metal ion contaminants on a surface, the method comprising:
(a) providing a device comprising a surface for performing a biochemical reaction;
(b) treating the surface with an EDTA solution;
(c) treating the surface with an alkaline solution comprising an oxidizing agent, wherein said solution is at a temperature between room temperature and 75° C. thereby removing metal ion or semi-metal ion contaminants bound to the surface.

37. A method for minimizing contamination with metal ions or semi-metal ions to a surface, the method comprising:
(a) providing a device comprising a surface;
(b) treating said surface with an alkaline solution comprising an oxidizing agent, wherein said solution is at a temperature between room temperature and 75° C.;
(d) treating said surface with an EDTA solution;
(e) repeating step (b) thereby minimizing contamination with metal ions or semi-metal ions on the surface of the device.
